(19) United States
(12) Patent Application Publication
(10) Pub. No.: US 2024/0325856 A1
(43) Pub. Date: Oct. 3, 2024

(54) USER EXPERIENCE PERSONALIZATION IN A CONNECTED FITNESS PLATFORM

(71) Applicant: Peloton Interactive, Inc., New York, NY (US)

(72) Inventors: Shoya YOSHIDA, New York, NY (US); Shayak BANERJEE, Ardsley, NY (US); Nganba MEETEI, New Providence, NJ (US); Natalia CHEN, New York, NY (US)

(21) Appl. No.: 18/622,377

(22) Filed: Mar. 29, 2024

Related U.S. Application Data
(60) Provisional application No. 63/492,881, filed on Mar. 29, 2023.

(51) Int. Cl. A63B 71/06; A63B 22/02; A63B 22/06; A63B 24/00; G06F 3/0482

(52) U.S. Cl. CPC A63B 71/0622; A63B 22/02; A63B 22/06; A63B 24/0062; A63B 2071/0625; A63B 2220/62; G06F 3/0482

(57) ABSTRACT

The systems and methods described herein facilitate an enhanced user experience within a connected fitness system. For example, the systems and methods can enhance and/or personalize a homescreen experience and interface for a user of a connected fitness platform. The homescreen experience, when personalized, can provide a user with enhanced or specifically tailored rows of content, such as recommended classes or activities, for selection by the user.

### CROSS REFERENCE TO RELATED APPLICATIONS

This application claims priority to U.S. Provisional Patent Application No. 63/492,881, filed on Mar. 29, 2023, entitled USER EXPERIENCE PERSONALIZATION IN A CONNECTED FITNESS PLATFORM, which is hereby incorporated by reference in its entirety.

### BACKGROUND

The world of connected fitness is an ever-expanding one. This world can include a user taking part in an activity (e.g., running, cycling, lifting weights, and so on), other users also performing the activity, and users doing other activities. The users may be utilizing a fitness machine (e.g., a treadmill, a stationary bike, a strength machine, a stationary rower, and so on), or may be moving through the world on a bicycle or other equipment.

The users can also be performing other activities that do not include an associated machine, such as running, strength training, yoga, stretching, hiking, climbing, and so on. These users can have wearable devices or mobile devices (e.g., heart rate monitors) that monitor the activity or performance of the users. The users can also perform the activity in front of a user interface (e.g., a display or device) presenting content associated with the activity, or outside of any displayed content.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), and so on.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIG. 2 is a block diagram illustrating example components of a recommendation system.

FIG. 3 is a diagram illustrating matrix factorization to determine instructor that are similar to one another.

FIG. 4 is a flow diagram illustrating a method for presenting content to a user of a connected fitness platform.

FIG. 5 is a flow diagram illustrating another method for presenting content to a user of a connected fitness platform.

FIGS. 6A-6B are diagrams illustrating example user interfaces for displaying rows of content via a homescreen of a connected fitness platform.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods facilitate an enhanced user experience within a connected fitness system. For example, the systems and methods can enhance and/or personalize a home screen experience (or, homescreen) and interface for a user of a connected fitness platform. The home screen experience, when personalized, can provide a user with enhanced or specifically tailored rows of content, such as recommended classes or activities, for selection by the user.

For example, a method may include accessing exercise class activity information for a user of a connected fitness platform, wherein each exercise class is associated with an instructor, applying matrix factorization to the exercise class activity information to identify a first set of instructors and a second set of instructors similar to the first set of instructors, and surfacing a user-selectable set of exercise classes via a user interface of the connected fitness platform, wherein the set of exercise classes includes exercise classes associated with the first set of instructors and the second set of instructors.

As another example, a method may include determining a musical artist of interest to a user, identifying musical artists similar to the musical artist of interest to the user based on applying matrix factorization to interactions between the user and exercise content presented during exercise activities previously performed by the user, and generating exercise class recommendations for the user that are based on the identified musical artists similar to the musical artist of interest to the user.

Thus, in various embodiments, the systems and methods personalize content for users/members of a connected fitness platform or other platform that presents diverse sets of content to users. Such personalization may enhance a user's experience when exercising or navigating various interfaces presented within a connected fitness platform, among other benefits.

Various embodiments of the systems and methods will now be described. The following description provides specific details for a thorough understanding and an enabling description of these embodiments. One skilled in the art will understand, however, that these embodiments may be practiced without many of these details.

Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

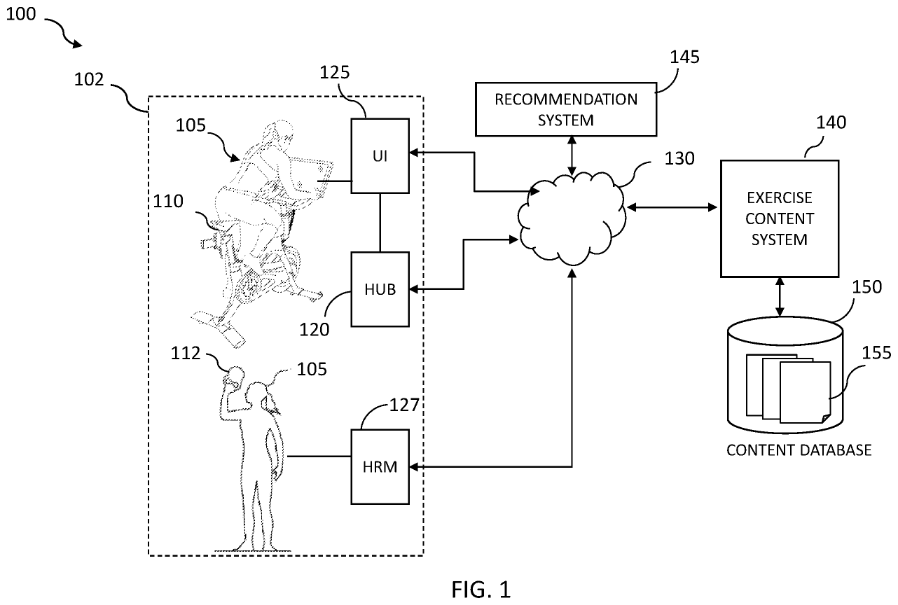

The technology described herein is directed, in some embodiments, to providing a user with an enhanced user experience (e.g., a personalized experience) when performing an exercise activity, such as an exercise activity as part of a connected fitness system or other exercise system. FIG. 1 is a block diagram illustrating a suitable network environment 100 for users of an exercise system.

The network environment 100 includes an activity environment 102, where a user 105 is performing an exercise activity, such as a cycling activity. In some cases, the user 105 can perform the activity with an exercise machine 110, such as exercise bicycle, a treadmill, a rowing machine, a stair climber, and so on. Further, the exercise activity performed by the user 105 can include a variety of different workouts, activities, actions, and/or movements not associated with a machine, such as movements associated with lifting weights 112 (as shown), stretching, doing yoga, pilates, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., streamed classes, dynamically changing video, audio, video games, instructional content, and so on) to the user 105 during an activity or workout.

The exercise machine 110 includes a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box or other similar device that outputs signals to a display, such as the user interface 125. Thus, the media hub 120 can operate to both capture images of the user 105 during an activity, while also presenting content (e.g., time-based or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

As described herein, the user interface 125 may present personalized recommendations to a user, based on the technology described herein. The personalized recommendations may be presented via a home screen of the platform or another screen or interface that provides a portal into the various types of classes, activities, or workouts provided by the platform. The user interface 125, therefore, may present specialized or personalized rows of content (e.g., different user-selectable classes or activities), as described herein.

In some cases, a heart rate monitor (HRM) 127 or other wearable device (e.g., smart watch, headphones, fitness trackers, and so on) can capture biometric information about the user 105, such as heart rate, movement information, sleep information, and so on. The HRM 127 can capture the user's heart rate and other information during machine- based activities and/or other activities, such as offline or class-based activities that do not utilize the exercise machine 110. In some cases, the exercise machine can include components configured to capture biometric information for the user 105, such as heart rate information.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) an exercise content system 140 over the network 130. In other cases, the media hub 120 controls the communication of content to/from the exercise content system 140 over the network 130 and presents the content to the user via the user interface 125.

The exercise content system 140, located at one or more servers remote from the user 105, can include various content libraries (e.g., classes, movements, tutorials, and so on) and perform functions to stream or otherwise send content to the machine 110, the media hub 120, and/or the user interface 125 over the network 130.

A content database 150 stores content 155 (e.g., video files) that presents a pre-recorded class to a user. The content can include images, video, and other visual information that present the class, music and other audio information to be played during the activity, and various overlay or augmentation information that is presented along with the audio/video content. Further, the database 150 can include various content libraries (e.g., classes, movements, tutorials, and so on) associated with the content presented to the user during a selected experience.

As described herein, a recommendation system 145 can include various components configured to boost recommendations and/or personalize a user or member experience within the environment 100, such as via a home screen presented via the user interface 125. Further details regarding the functionality of the recommendation system 145 are described herein.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 130 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 130 and are shown as separate connections, these connections can be any kind of local, wide area, wired, or wireless network, public or private.

Further, any or all components depicted in the Figures described herein can be supported and/or implemented via one or more computing systems, services (e.g., cloud instances), or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of the Personalized Home Screen User Experience

Often, a user accesses content, such as exercise classes, via an on-demand library and a homescreen, or entry screen. The on-demand library is a catalog view of all classes available within the connected fitness platform. The user may apply various filters and sorting to find classes and/or may perform free-form text searches to find classes. The homescreen is the first screen that the user (or member) encounters upon logging in and serves as a gateway into the user's connected fitness experience. Via the homescreen, the user may scroll vertically to see various rows of content as well as horizontally within each row to see specific classes or activities for each of the rows.

Given a connected fitness platform having a significant number of classes or content (e.g., tens of thousands of classes, hundreds of class types, many (e.g., 50+) instructors, many fitness disciplines, thousands of artists and music, and so on), it can be difficult and often a bit overwhelming for a user to find content they may enjoy.

The homescreen can be a useful mechanism for surfacing and/or recommending new or unknown content to the user. For example, in addition to providing a mix of personalized modules, manually curated content, and promotional banners for new experiences, the homescreen can also present personalized rows of content recommendations to further customize and enhance the experience for the user.

As described herein, in some embodiments, the systems and methods perform various methods or operations to personalize a user experience within or using a connected fitness platform. For example, the systems and methods may utilize collaborative filtering or other similar techniques to identify, recommend, and/or surface exercise classes for users based on determining instructors that are similar to one another (with respect to a specific user or users), music that is similar to other music, and so on.

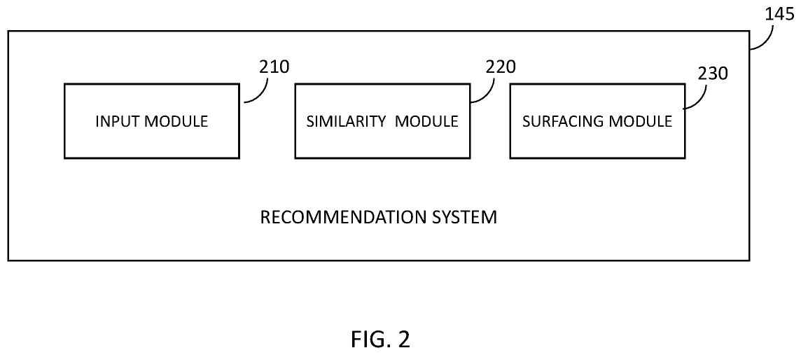

FIG. 2 is a block diagram illustrating example components of the recommendation system 145. As described herein, the recommendation system 145 may include components and/or modules that enable a connected fitness platform to identify exercise classes and other content for users by identifying classes that are associated with instructors or music that is similar to the instructions or music known to be preferred or of interest to the users.

In some embodiments, the recommendation system 145 includes an input module 210 that is configured and/or programmed to receive or access historical activity information for the user of the connected fitness platform. The activity information can include exercise classes previously performed by the user, where each exercise class is associated with a specific instructor of the connected fitness platform (or specific music, such as songs, artists, types, and so on).

In some embodiments, the recommendation system 145 includes a similarity module 220 that is configured and/or programmed to apply matrix factorization to the historical activity information to identify instructors that are similar to one another. For example, the system 145 employs collaborative filtering, such as by using matrix factorization, to determine one or more highest engagement instructors that are most engaged with by the user of the connected fitness platform, and one or more similar instructors that are related to the one or more highest engagement instructors based on the applied matrix factorization.

As described herein, the system 145 can identify classes of interest to the user that are associated with instructors they have rarely or never interacted with previously. The similarity module 220 applies collaborative filtering to identify those instructors. To identify similar instructors, the similarity module 220 utilizes collaborative filtering. For example, the module 220 can employ matrix factorization, which decomposes a user-item interaction matrix into a product of two lower dimension rectangular matrices.

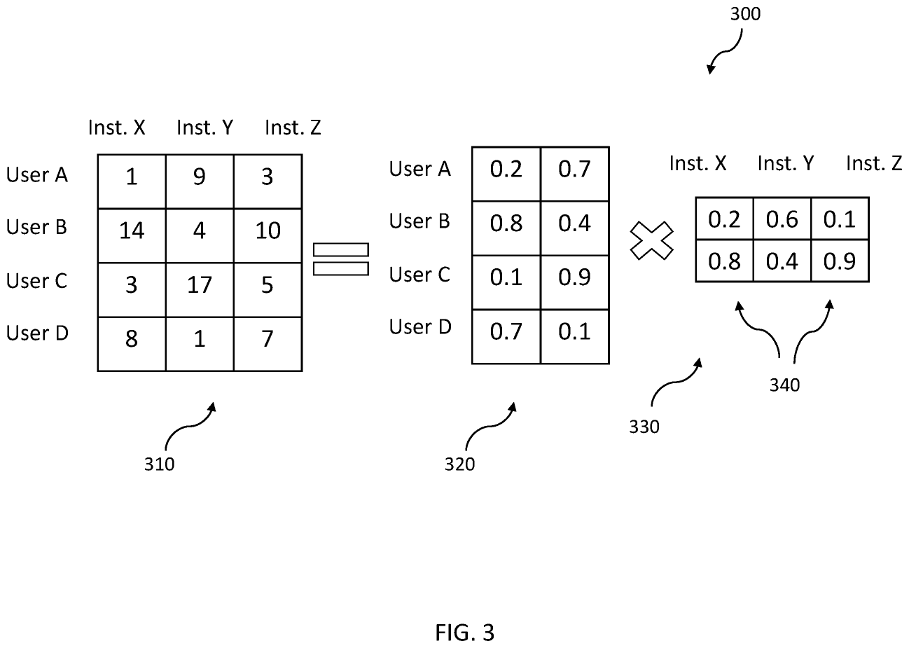

FIG. 3 is a diagram 300 illustrating matrix factorization to determine instructor that are similar to one another. The diagram 300 includes a user-instructor matrix 310, where each “item” in the matrix is an instructor of the connected fitness platform. The user-instructor matrix 310 includes ratings for each instructor, The module 220 can identify other users that provide similar ratings, and use those ratings to determine similar instructors, as follows.

The similarity module 220 fits a matrix factorization model on user workout histories, where the “items” are instructors, and utilizes the learned factors for each instructor to find similar instructors to that instructor. For example, the entries of the user-instructor matrix 310 are the number of times each user has interacted with the instructor in their recent workout history. Thus, using the collaborative filtering approach, the module 220 can directly capture which instructors its users think are similar, and continuously adapt to trends over time.

The module 220 then applies cosine similarity, determining the product of a user matrix 320 and an instructor matrix 330. In some cases, the instructor matrix may be specific to an exercise type (since many instructors are associated with different class types). The module 22 then identifies the similar instructors 340 (e.g., Instructor X and Instructor Z).

For example, the module 220 applies matrix factorization includes determining rating vectors for instructors of the connected fitness platform (e.g., of a common discipline or class type), generates a rating matrix that maps the instructors of the connected fitness platform with their determined rating vectors, and determines the one or more similar instructors are related to the one or more highest engagement instructors using cosine similarity.

Returning back to FIG. 2, in some embodiments, the recommendation system 145 includes a surfacing module 230 that is configured and/or programmed to display, or cause to display, a user-selectable set of exercise classes via a user interface of the connected fitness platform. The user-selectable set of exercises can include at least one exercise class associated with the one or more highest engagement instructors, and at least one exercise class associated with the one or more similar instructors that are related to the one or more highest engagement instructors based on the applied matrix factorization.

For example, the surfacing module 230 can generate rows of recommended classes for a homescreen that is specific to a user of the connected fitness platform. The rows can include classes known to be of interest to the user (e.g., classes associated with the user's most watched instructor), as well as classes determined by the system 145 to be potentially of interest to the user, because the classes are associated with instructors determined to be similar to the user's most watched instructor.

In some embodiments, after determining the similar instructors, the recommendation system 145 utilizes a ranking and filtering approach, and filters available classes to identify classes that are only associated with (e.g., taught by) the determined similar instructors. Thus, the system 145 can identify and select for a given user classes associated with the determined similar instructors that may be preferred by the user based on other attributes (e.g., class type, duration, music choices, effort level, and so on).

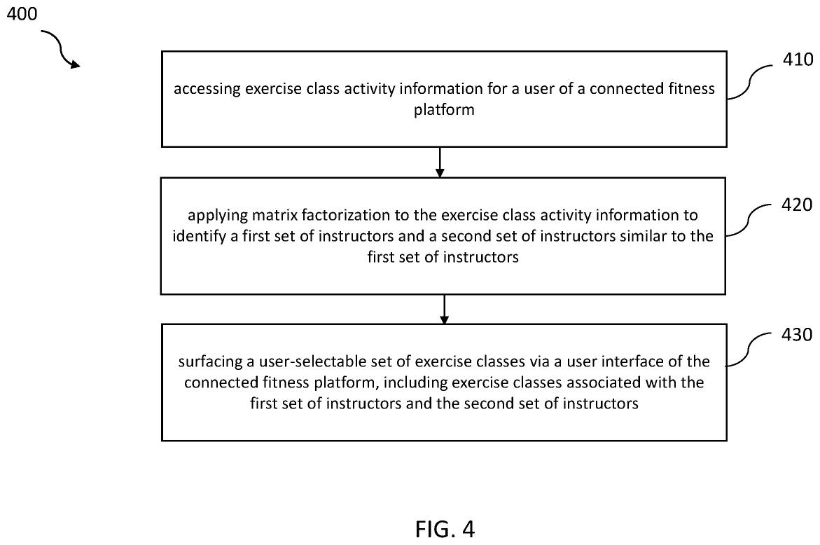

FIG. 4 is a flow diagram illustrating a method 400 for presenting content to a user of a connected fitness platform. The method 400 may be performed by the recommendation system 145 and, accordingly, is described herein merely by way of reference thereto. It will be appreciated that the method 400 may be performed on any suitable hardware.

In operation 410, the recommendation system 145 accesses exercise class activity information for a user of a connected fitness platform, where each exercise class is associated with an instructor (e.g., taught by an instructor). For example, the input module 210 receives or accesses historical activity information for the user of the connected fitness platform, such as the user's most watched or utilized instructors, the user's most watched classes, and so on.

In operation 420, the recommendation system 145 applies matrix factorization to the exercise class activity information to identify a first set of instructors and a second set of instructors similar to the first set of instructors. For example, the similarity module 220 employs collaborative filtering to determine one or more highest engagement instructors that are most engaged with by the user of the connected fitness platform, and one or more similar instructors that are related to the one or more highest engagement instructors based on the applied matrix factorization.

In operation 430, the recommendation system 145 surfaces, or causes to surface, a user-selectable set of exercise classes via a user interface of the connected fitness platform. For example, the surfacing module 230 may render, update, and/or cause a specialized row on a homescreen for the user to present a set of exercise classes that includes exercise classes associated with the first set of instructors and the second set of instructors (e.g., one or more instructors deemed to be “similar” to the user's favorite or most watched instructors).

In addition to identifying and recommending classes based on determining similar instructors, the recommendation system 145 may apply the approaches described herein to identify musical artists that are similar to a user's favorite or most engaged with musical artists, and surface exercise classes associated with those musical artists. Often, the music associated with an exercise class drives engagement to that exercise class, and, therefore, discovering other exercise classes via a determination of similar musical artists, using the collaborative filtering approaches described herein, can enable a connected fitness platform to enhance a user's experience and engagement within the platform.

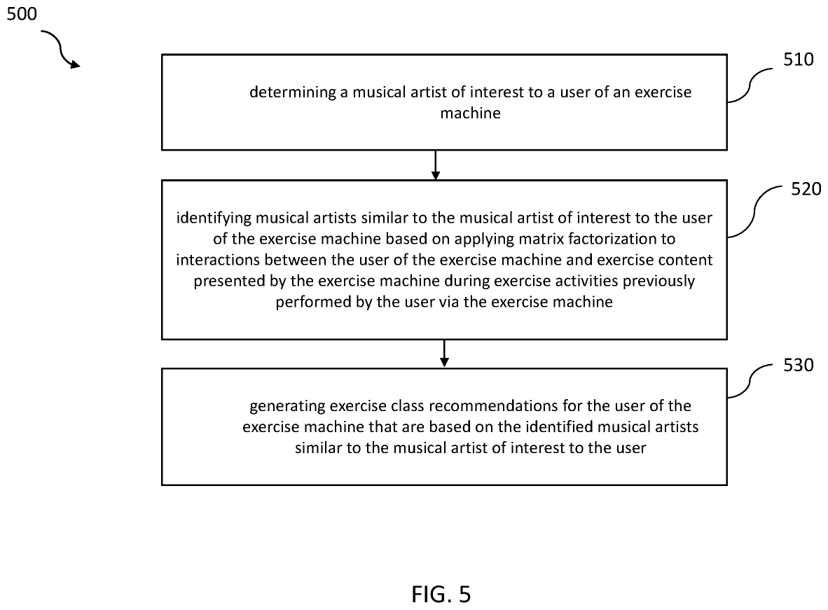

FIG. 5 is a flow diagram illustrating a method 500 for presenting content to a user of a connected fitness platform. The method 500 may be performed by the recommendation system 145 and, accordingly, is described herein merely by way of reference thereto. It will be appreciated that the method 500 may be performed on any suitable hardware.

In operation 510, the recommendation system 145 determines a musical artist of interest to a user of an exercise machine. For example, the system may receive an indication that a user has explicitly “liked” or otherwise indicated an interest in a song or musical artist during an exercise class (e.g., selected a “heart” icon), may receive a rating from the user about the class or other classes, may receive an indication that a user has taken an artist-specific or-themed class, and so on.

Thus, the system 145 may determine a user has an affinity for a specific musical artist by receiving explicit feedback from a user of an exercise machine for the musical artist, determining the user has participated in a threshold number of exercise classes that include music by the musical artist, determining the user has participated in an exercise class that features the musical artist, and so on.

In operation 520, the recommendation system 145 identifies musical artists similar to the musical artist of interest to the user of the exercise machine based on applying matrix factorization to interactions between the user of the exercise machine and exercise content presented by the exercise machine during exercise activities previously performed by the user via the exercise machine.

For example, as described herein, the system 145, via the similarity module 220, utilizes collaborative filtering to learn the affinities/interests of the user on a musical artist level. Using the matrix 300 depicted in FIG. 3, similarity module 220 may input values based on a weighted sum of feedback from various possible feedback sources from the user (e.g., 10 points for a specific artist if the user explicitly “hearts” a song, 1 point for each artist-specific class, and so on). Once user-artist affinity scores are determined, the module 220 aggregates the scores, for each playlist attached to or associated with an exercise class, to determine an overall user-playlist affinity score for the exercise class.

The module 220 may then scale the score using duration affinities (e.g., class length preferences), and identifies class recommendations to be surfaced to the user. Thus, the recommendation system 145 may learn artist-level affinities to pinpoint a featured artist that the user likes, or is predicted to like, within the homescreen for the user.

In operation 530, the recommendation system 145 generates exercise class recommendations for the user of the exercise machine that are based on the identified musical artists similar to the musical artist of interest to the user. For example, the surfacing module 230 may generate rows of recommended classes for a homescreen that is specific to a user of the connected fitness platform, with explanations that indicate the reasoning behind certain recommended classes (e.g., “users who like Artist A also like Artist C).

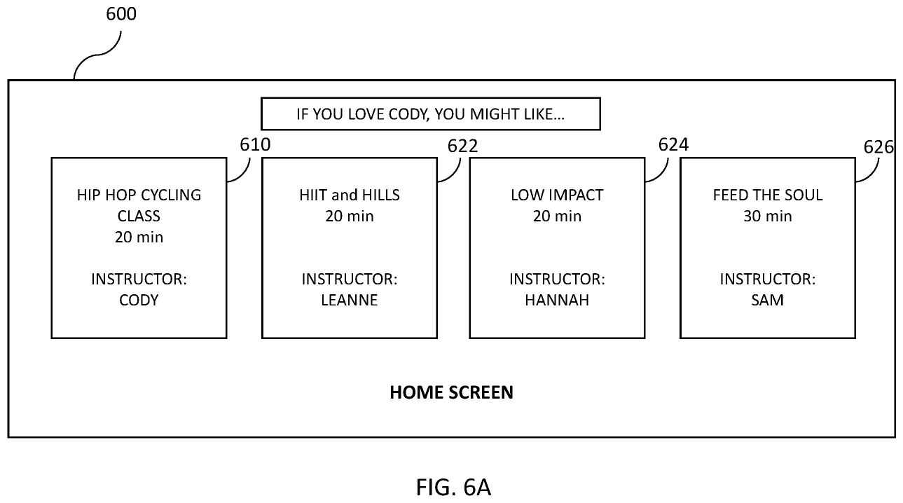

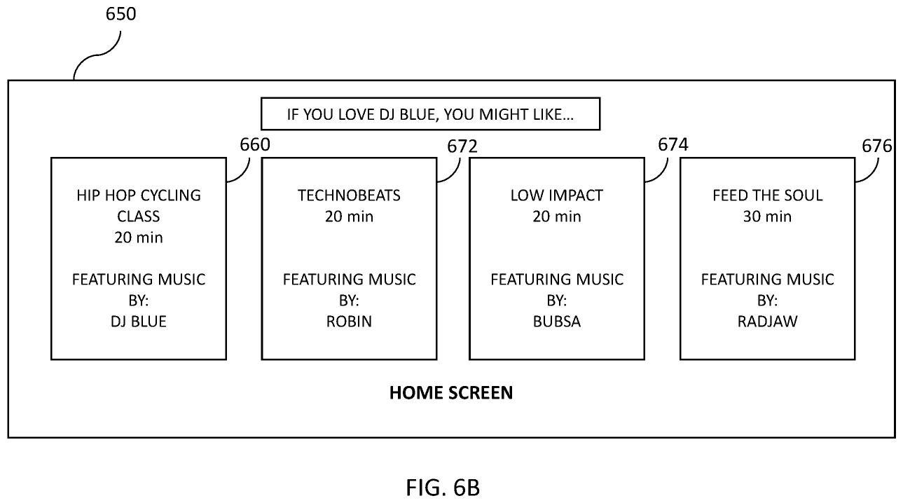

As described herein, the recommendation system 145 may render or generate rows of recommended content to present to users via a homescreen of a connected fitness platform. FIGS. 6A-6B depict example homescreens rendered for a user using the collaborative filtering described herein.

For example, FIG. 6A depicts an example homescreen 600 that presents a row of exercise classes, including an exercise class 610 for an instructor (e.g., “Cody”) known to be of interest to the user, and exercise classes 622, 624, 626 having instructors determined to be similar to Cody using the collaborative filtering techniques described herein. The user may then select a class (e.g., class 626, led by “Sam”) having an instructor previously unknown, or unengaged with, by the user.

As another example, FIG. 6B depicts an example homescreen 650 that presents a row of exercise classes, including an exercise class 660 associated with a musical artist (e.g., “DJ BLUE”) known to be of interest to the user, and exercise classes 672, 6274, 676 having music from artists determined to be similar to the liked musical artist using the collaborative filtering techniques described herein. The user may then select a class (e.g., class 676, led by “Radjaw”) having an instructor previously unknown, or unengaged with, by the user.

In some cases, the system 145 may utilize various heuristics when presented class recommendations. For example, the homescreens 600, 650 may include graphical elements that enable a user to request certain groups of classes associated with instructors and/or musical artists determined to be similar using the technology described herein. For example, the system 145 may enable a query or request of “show me the most popular classes by [similar instructor],” “show me the most recent classes from [similar instructor],” or “show me other classes that have music from [similar artist].” The system 145 then render or update presented options to the user based on the query or request.

Thus, in various embodiments, the technology described herein facilitates user engagement with a large collection of exercise classes provided by a connected fitness platform. The technology may employ collaborative filtering to determining similarities between instructors and/or music associated with the classes and identify classes potentially of interest to a user that the user would normally not select or be drawn to using other filtering or matching techniques. The connected fitness platform may generate rows of such content via a homescreen or other entry portal, enabling the user to quickly find, select, and/or engage with a broader set of content (e.g., exercise classes or activities), enhancing the user's experience with the platform, among other benefits.

### Example Embodiments of the Technology

In some embodiments, a system for surfacing content to a user of a connected fitness platform includes an input module that receives historical activity information for the user of the connected fitness platform, wherein the historical activity information includes exercise classes previously performed by the user, and wherein each exercise class is associated with a specific instructor of the connected fitness platform, a similarity module that applies matrix factorization to the historical activity information to identify one or more highest engagement instructors that are most engaged with by the user of the connected fitness platform and one or more similar instructors that are related to the one or more highest engagement instructors based on the applied matrix factorization, and a surfacing module that displays a user-selectable set of exercise classes via a user interface of the connected fitness platform, wherein the set of exercise classes includes at least one exercise class associated with the one or more highest engagement instructors, and at least one exercise class associated with the one or more similar instructors that are related to the one or more highest engagement instructors based on the applied matrix factorization.

In some cases, applied matrix factorization includes determining rating vectors for instructors of the connected fitness platform, generating a rating matrix that maps the instructors of the connected fitness platform with their determined rating vectors, and determining the one or more similar instructors are related to the one or more highest engagement instructors using cosine similarity.

In some cases, determining the rating vectors for the instructors of the connected fitness platform includes determining the rating vectors for all instructors associated with exercise classes of a common exercise discipline.

In some cases, the input module receives information for the exercise classes previously performed by the user that identifies a duration of each of the exercise classes and music selections for each of the exercise classes.

In some cases, the surfacing module displays the user-selectable set of exercise classes via a homescreen interface via which the user accesses the connected fitness platform.

In some cases, the surfacing module displays the user-selectable set of exercise classes via one or more rows of recommended class content that include the user-selectable set of exercise classes.

In some cases, the user interface is a display screen that is part of a treadmill via which the user performs exercise activities during exercise classes streamed by the connected fitness platform.

In some cases, the user interface is a display screen that is part of an exercise bicycle via which the user performs exercise activities during exercise classes streamed by the connected fitness platform.

In some cases, the user interface is a display screen that is part of a mobile device via which the user consumes exercise content streamed by the connected fitness platform.

In some embodiments, a method includes accessing exercise class activity information for a user of a connected fitness platform, wherein each exercise class is associated with an instructor, applying matrix factorization to the exercise class activity information to identify a first set of instructors and a second set of instructors similar to the first set of instructors, and surfacing a user-selectable set of exercise classes via a user interface of the connected fitness platform, wherein the set of exercise classes includes exercise classes associated with the first set of instructors and the second set of instructors.

In some cases, applying matrix factorization to the exercise class activity information includes determining the first set of instructors is similar to the second set of instructors using cosine similarity.

In some cases, applying matrix factorization to the exercise class information includes determining rating vectors for instructors of the connected fitness platform, generating a rating matrix that maps the instructors of the connected fitness platform with their determined rating vectors, and determining the first set of instructors is similar to the second set of instructors using cosine similarity.

In some cases, the exercise class activity information includes duration information for the exercise classes and music selections for the exercise classes.

In some cases, the user interface of the connected fitness platform is a homescreen interface via which the user accesses the connected fitness platform.

In some cases, the user interface of the connected fitness platform includes one or more rows of recommended class content that include the user-selectable set of exercise classes.

In some cases, the user interface of the connected fitness platform is a display screen that is part of a treadmill via which the user performs exercise activities during exercise classes streamed by the connected fitness platform.

In some cases, the user interface of the connected fitness platform is a display screen that is part of an exercise bicycle via which the user performs exercise activities during exercise classes streamed by the connected fitness platform.

In some cases, the user interface of the connected fitness platform is a display screen that is part of a mobile device via which the user consumes exercise content streamed by the connected fitness platform.

In some embodiments, a method includes determining a musical artist of interest to a user of an exercise machine, identifying musical artists similar to the musical artist of interest to the user of the exercise machine based on applying matrix factorization to interactions between the user of the exercise machine and exercise content presented by the exercise machine during exercise activities previously performed by the user via the exercise machine, and generating exercise class recommendations for the user of the exercise machine that are based on the identified musical artists similar to the musical artist of interest to the user.

In some cases, determining the musical artist of interest to the user of the exercise machine includes receiving explicit feedback from the user of the exercise machine for the musical artist of interest to the user, or determining the user has participated in a threshold number of exercise classes that include music by the musical artist of interest to the user, and/or determining the user has participated in an exercise class that features the musical artist of interest to the user.

### Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system for surfacing content to a user of a connected fitness platform, the system comprising:
an input module that receives historical activity information for the user of the connected fitness platform,
wherein the historical activity information includes exercise classes previously performed by the user, and
wherein each exercise class is associated with a specific instructor of the connected fitness platform;
a similarity module that applies matrix factorization to the historical activity information to identify:
one or more highest engagement instructors that are most engaged with by the user of the connected fitness platform; and
one or more similar instructors that are related to the one or more highest engagement instructors based on the applied matrix factorization; and
a surfacing module that displays a user-selectable set of exercise classes via a user interface of the connected fitness platform,
wherein the set of exercise classes includes:
at least one exercise class associated with the one or more highest engagement instructors, and
at least one exercise class associated with the one or more similar instructors that are related to the one or more highest engagement instructors based on the applied matrix factorization.

2. The system of claim 1, wherein the applied matrix factorization includes:
determining rating vectors for instructors of the connected fitness platform;
generating a rating matrix that maps the instructors of the connected fitness platform with their determined rating vectors; and
determining the one or more similar instructors are related to the one or more highest engagement instructors using cosine similarity.

3. The system of claim 2, wherein determining the rating vectors for the instructors of the connected fitness platform includes determining the rating vectors for all instructors associated with exercise classes of a common exercise discipline.

4. The system of claim 1, wherein the input module receives information for the exercise classes previously performed by the user that identifies a duration of each of the exercise classes and music selections for each of the exercise classes.

5. The system of claim 1, wherein the surfacing module displays the user-selectable set of exercise classes via a homescreen interface via which the user accesses the connected fitness platform.

6. The system of claim 1, wherein the surfacing module displays the user-selectable set of exercise classes via one or more rows of recommended class content that include the user-selectable set of exercise classes.

7. The system of claim 1, wherein the user interface is a display screen that is part of a treadmill via which the user performs exercise activities during exercise classes streamed by the connected fitness platform.

8. The system of claim 1, wherein the user interface is a display screen that is part of an exercise bicycle via which the user performs exercise activities during exercise classes streamed by the connected fitness platform.

9. The system of claim 1, wherein the user interface is a display screen that is part of a mobile device via which the user consumes exercise content streamed by the connected fitness platform.

10. A method, comprising:
accessing exercise class activity information for a user of a connected fitness platform,
wherein each exercise class is associated with an instructor;
applying matrix factorization to the exercise class activity information to identify a first set of instructors and a second set of instructors similar to the first set of instructors; and
surfacing a user-selectable set of exercise classes via a user interface of the connected fitness platform,
wherein the set of exercise classes includes exercise classes associated with the first set of instructors and the second set of instructors.

11. The method of claim 10, wherein applying matrix factorization to the exercise class activity information includes determining the first set of instructors is similar to the second set of instructors using cosine similarity.

12. The method of claim 10, wherein applying matrix factorization to the exercise class information includes:
determining rating vectors for instructors of the connected fitness platform;
generating a rating matrix that maps the instructors of the connected fitness platform with their determined rating vectors; and
determining the first set of instructors is similar to the second set of instructors using cosine similarity.

13. The method of claim 10, wherein the exercise class activity information includes duration information for the exercise classes and music selections for the exercise classes.

14. The method of claim 10, wherein the user interface of the connected fitness platform is a homescreen interface via which the user accesses the connected fitness platform.

15. The method of claim 10, wherein the user interface of the connected fitness platform includes one or more rows of recommended class content that include the user-selectable set of exercise classes.

16. The method of claim 10, wherein the user interface of the connected fitness platform is a display screen that is part of a treadmill via which the user performs exercise activities during exercise classes streamed by the connected fitness platform.

17. The method of claim 10, wherein the user interface of the connected fitness platform is a display screen that is part of an exercise bicycle via which the user performs exercise activities during exercise classes streamed by the connected fitness platform.

18. The method of claim 10, wherein the user interface of the connected fitness platform is a display screen that is part of a mobile device via which the user consumes exercise content streamed by the connected fitness platform.

19. A non-transitory computer-readable medium whose contents, when executed by a computing system, cause the computing system to perform a method, the method comprising:
determining a musical artist of interest to a user of an exercise machine;
identifying musical artists similar to the musical artist of interest to the user of the exercise machine based on applying matrix factorization to interactions between the user of the exercise machine and exercise content presented by the exercise machine during exercise activities previously performed by the user via the exercise machine; and
generating exercise class recommendations for the user of the exercise machine that are based on the identified musical artists similar to the musical artist of interest to the user.

20. The computer-readable medium of claim 19, wherein determining the musical artist of interest to the user of the exercise machine includes:
receiving explicit feedback from the user of the exercise machine for the musical artist of interest to the user; or
determining the user has participated in a threshold number of exercise classes that include music by the musical artist of interest to the user; or
determining the user has participated in an exercise class that features the musical artist of interest to the user.
